(19) United States
(12) Patent Application Publication
(10) Pub. No.: US 2008/0076700 A1
(43) Pub. Date: Mar. 27, 2008

(54) Method for the Preparation of Oxime, Thiazolidine, Dithiane, Dithiolane, or Hydrazone Linked Analogues of Growth Hormone

(75) Inventors: Florencio Zaragoza Dorwald, Smorum (DK); Christine Bruun Schiodt, Bronshoj (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/577,213

(22) PCT Filed: Oct. 18, 2005
(86) PCT No.: PCT/EP05/55335

§ 371 Date: Nov. 15, 2007

(30) Foreign Application Priority Data
Oct. 18, 2004 (DK) PA 2004 01588
Dec. 20, 2004 (EP) 04106725.7
Jan. 21, 2005 (DK) PA 2005 00107

(51) Int. Cl. A61K 38/04

(52) U.S. Cl. 514/2

(57) ABSTRACT

A method for the production of conjugated growth hormone is provided, wherein a GH derived aldehyde or ketone is reacted with an alkoxyamine, hydrazine or 2-aminothiol compound at acid pH in the presence of a dipolar solvent.

### FIELD OF THE INVENTION

The present invention relates to methods of preparing conjugated growth hormone (GH), wherein the conjugating moiety is bonded to the growth hormone by means of an oxime, thiazolidine, dithiane, dithiolane or hydrazone linkage.

### BACKGROUND OF THE INVENTION

The covalent attachment of polyethylene glycol (PEG), fatty acids, or other compounds to peptides and proteins with the aim of obtaining conjugates with improved pharmacological properties is a well-established strategy (Zobel et al., Bioorg. Med. Chem. Lett. 2003, 13, 1513-1515). Covalent attachment of compounds to proteins is generally performed by acylation (amide-bond formation with lysine side-chains or with the N-terminal amino acid) or by a condensation reaction of a suitable alkoxylamine, hydrazine or 2-aminothiol compound with a protein-derived ketone or aldehyde to yield an oxime, a hydrazone, or a thiazolidine, respectively (Shao and Tam, J. Am. Chem. Soc. 1994, 117, 3893-3899). These reactions, however, can only be conducted under conditions under which the protein, the resulting conjugated protein, and the conjugating reagent are dissolved.

It has been shown (R. Clark et al. J. Biol. Chem. 1996, 271, 21969-21977) that the acylation of human growth hormone (hGH) with PEG-derived acylating reagents leads to hGH conjugates with improved pharmacological properties. Regioselective acylation of hGH is, however, difficult, because this protein contains seven lysine residues of similar reactivity, and mixtures of products usually result. The single components of these mixtures are difficult to isolate, and will usually be obtained in low yield and purity only. It is therefore desirable to find a method for the conversion of a growth hormone-derived ketone or aldehyde by reaction with a suitable alkoxylamine, hydrazine, aminothiol or dithiol compound into an oxime, hydrazone, or thiazolidine linked conjugate. If only one ketone or aldehyde functionality is present in the starting growth hormone, conjugation will only take place at one site in the growth hormone, and a highly homogeneous product, suitable as therapeutic agent for humans may result.

Growth hormone is a key hormone involved in the regulation of not only somatic growth, but also in the regulation of metabolism of proteins, carbohydrates and lipids. The major effect of growth hormone is to promote growth. Human growth hormone (hGH) is a 191 amino acid residue protein with the sequence

(SEQ ID NO:1)

FPTIPLSRLFDNAMLRAHRLHQLAFDTYQEFEEAYIPKEQKYSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGE.

Shao and Tam have shown in J.Am.Chem.Soc., 1995, 117, 3893-3899 that the reaction between a VA20 derived alkoxyamine, hydrazine or 2-aminothiol and a glyoxylyl-lysinyl peptide has the highest rate at pH 4.2, 4.7 and 4.0, respectively, and that addition of organic solvents further increases this rate. VA20 is a highly basic, 20 amino acid residue peptide derived from the urface protein of feline immunodeficiency virus, and it has an isoelectric point (pl, the pH at which its solubility in water is lowest) around 11.4.

The isoelectric point of hGH is 5.1, and if e.g. an oximation of a hGH-derived aldehyde or ketone is attempted at pH around 4 (e.g. in the presence of acetic acid) precipitation of the protein usually occurs, and no high yield of oxime can be obtained. This precipitation will be further promoted in the presence of PEG, because this material has a high affinity for water and induces the precipitation of proteins.

### SUMMARY OF THE INVENTION

The inventors have found that the precipitation of reactants and/or product in the reaction between (a) a GH derived aldehyde or ketone and (b) a suitable alkoxyamine, hydrazine, aminothiol or dithiol compound can be avoided while maintaining a useful reaction rate by running the reaction at acidic pH in the presence of a dipolar solvent.

Accordingly, the invention provides a method for the production of a conjugated GH with improved pharmacological properties compared to the un-conjugated growth hormone compound (parent growth hormone), the method comprising the reaction between a GH derived aldehyde or ketone and a alkoxyamine, hydrazine, aminothiol or dithiol compound at pH 1-7 in the presence of a dipolar solvent. This reaction results in a conjugated GH wherein the conjugating moiety is linked to GH via an oxime, thiazolidine, dithiane, dithiolane or hydrazone linkage.

The invention also provides conjugated GH compounds with improved pharmacological properties.

### DEFINITIONS

In the present context, “growth hormone” (GH) is intended to indicate a protein which exhibits growth hormone activity as determined in assay I herein. A protein which exhibits an activity above 20%, such as above 40%, such as above 60%, such as above 80% of that of hGH in said assay is defined as a growth hormone.

The term “radical” or “biradical” is intended to indicate a molecular fragment with one or two unpaired electrons, respectively. Such a fragment may be formally generated by removingone (e.g., a hydrogen) or two atoms or groups of atoms (e.g., a hydroxyl group) by homolytic bond cleavage, i.e. a bond cleavage, in which each of the two resulting fragments contains one of the two electrons which formed the original bond.

As used herein, “hGH(141)” means a radical formed by formal removal of the CONH₂-group from glutamine(141) in hGH, “hGH(40)” means a radical formed by formal removal of the CONH₂-group from glutamine(40) in hGH, and “hGH(40,141)” means a radical formed by formal removal of the CONH₂-groups from glutamine(40) and glutamine(141) in hGH. hGH(40/141) means a radical formed by formal removal of the CONH₂-groups from glutamine(40) and/or glutamine(141) in hGH, encompassing mixtures of two or more of hGH(40), hGH(141), and (hGH(40,141).

In the present context, a protein is intended to indicate a sequence of two or more amino acids joined by peptide bonds, wherein said amino acids may be natural or un-natural. It is to be understood that the term is also intended to include proteins which have been derivatized, e.g. by the attachment of lipophilic groups, PEG or prosthetic groups.

The term “cibacronyl” means the radical sketched below, or any salt or solvate of it

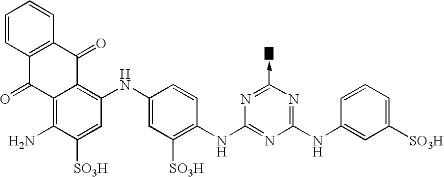

The term “dipolar solvent” refers to a solvent with a dielectric constant larger than 6.0.

The term “PEG” or “Peg”, used interchangeably herein, means a polydisperse or monodisperse diradical of the structure

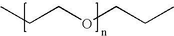

wherein n is an integer larger than 1, and its molecular weight is between approximately 100 and approximately 1,000,000 Da.

Thus, PEG or Peg is intended to indicate poly (ethylene glycol) as well as poly (ethylene glycol) mono alkyl ether, wherein alkyl in this context is intended to indicate C₁₋₆alkyl, such as methyl, ethyl, propyl, butyl, pentyl and hexyl. As an example, mPEG(XX k) represents a poly(ethylene glycol) monomethylether with a molecular weight of approximately XX kD. By way of example, mPEG(30k) is intended to indicate poly(ethylene glycol) monomethylether with a molecular weight of approximately 30 kD, i.e. containing approximately 680±100 ethylene glycol units. The molecular weight distribution of this polymer may vary from batch to batch. PEG(XX k) refers either to linear poly(ethylene glycol) or poly (ethylene glycol) mono alkyl ether, or to branched poly(ethylene glycol) or poly (ethylene glycol) mono alkyl ether.

The term “mPEG” or “mPeg”, used interchangeably herein, means a polydisperse or monodisperse radical of the structure

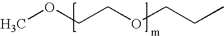

wherein m is an integer larger than 1. Thus, an mPEG wherein m is 90 has a molecular weight of 3991 Da, i.e. approx 4 kDa. Likewise, an mPEG with an average molecular weight of 20 kDa has an average m of 454. Due to the process for producing mPEG these molecules often have a distribution of molecular weights. This distribution is described by the polydispersity index.

Due to this distribution of m, mPEG with a molecular weight of 20 kDa may also be referred to as MeO-(CH₂CH₂O)₄₀₀₋₅₀₀, mPEG with a molecular weight of 30 kDa may also be referred to as MeO-(CH₂CH₂O)₆₀₀₋₇₀₀, and mPEG with a molecular weight of 40 kDa may also be referred to as MeO-(CH₂CH₂O)₈₅₀₋₉₅₀. The heavier mPEG chains may be difficult to prepare as a single chain molecule, and they are thus made as branched mPEG. Notably, mPEG with a molecular weight of 40 kDa may be achieved with as a branched mPEG comprising to arms of 20 kDa each.

The term “polydispersity index” as used herein means the ratio between the weight average molecular weight and the number average molecular weight, as known in the art of polymer chemistry (see e.g. “Polymer Synthesis and Characterization”, J. A. Nairn, University of Utah, 2003). The polydispersity index is a number which is greater than or equal to one, and it may be estimated from Gel Permeation Chromatographic data. When the polydispersity index is 1, the product is monodisperse and is thus made up of compounds with a single molecular weight. When the polydispersity index is greater than 1 it is a measure of the polydispersity of that polymer, i.e. how broad the distribution of polymers with different molecular weights is.

The use of for example “mPEG20000” in formulas, compound names or in molecular structures indicates an mPEG residue wherein mPEG is polydisperse and has a molecular weight of approximately 20 kDa.

The polydispersity index typically increases with the molecular weight of the PEG or mPEG. When reference is made to 20 kDa PEG and in particular 20 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 30 kDa PEG and in particular 30 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 40 kDa PEG and in particular 40 kDa mPEG it is intended to indicate a compound (or in fact a mixture of compounds) with a polydisperisty index below 1.06, such as below 1.05, such as below 1.04, such as below 1.03, such as between 1.02 and 1.03.

The term “arylene” as used herein is intended to indicate the bi-radical derived from carbocyclic aromatic ring systems comprising one or more rings, such as benzene, biphenyl, naphthene, anthracenene, phenanthrene, fluorene, indene, pentalene and azulene. Arylene is also intended to include the bi-radicals derived from the partially hydrogenated derivatives of the multi-ring carbocyclic systems enumerated above, wherein at least one ring is aromatic. Examples of such partially hydrogenated derivatives include 1,2,3,4-tetrahydronaphthene and 1,4-dihydronaphthene. Examples of arylene include 1,2-phenylene, 1,3-phenylene, 1,4-phenylene, 1,2-naphthylene, 1,4-naphthylene, 4,4′-biphenylene, 4,4″-30 terphenylene and 4,4″′-quaterphenylene.

The term “heteroarylene” as used herein is intended to indicate bi-radicals derived from heterocyclic aromatic ring systems containing one or more heteroatoms selected from nitrogen, oxygen and sulphur, such as furane, thiophene, pyrrole, oxazole, thiazole, imidazole, isoxazole, isothiazole, 1,2,3-triazole, 1,2,4-triazole, pyran, pyridine, pyridazine, pyrimidpyrimidine, pyrazine, 1,2,3-triazine, 1,2,4-triazine, 1,3,5- triazine, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, tetrazole, thiadiazine, indole, isoindole, benzofurane, benzothiophen, indazole, benzimidazole, benzthiazole, benzisothiazole, benzoxazole, benzisoxazole, purine, quinazoline, quinolizine, quinoline, isoquinoline, quinoxaline, naphthyridine, pteridine, carbazole, azepine, diazepine, acridine and the like. The term is also intended to include partially hydrogenated derivatives of the multi-ring heterocyclic systems enumerated above, provided at least one ring comprising a hetero atom is aromatic. Examples of such partially hydrogenated derivatives include 2,3-dihydrobenzofurane, pyrroline, pyrazoline, indoline, oxazolidine, oxazoline and oxazepine. Examples of “heteroarylene” include 1,2,4-pyrazol-2,5-diyl, imidazol-1,2-diyl, thiazol-2,4-diyl, (4-phenylimidazole)-4,1′-diyl and (3,5-diphenyl-1,2,4-oxadiazole)-4,4″-diyl.

In the present context, an alkoxyamine compound is intended to indicate a compound comprising an alkoxyamine (—O—NH₂) moiety.

In the present context, a hydrazine compound is intended to indicate a compound comprising a hydrazine (—N—NH₂) moiety.

In the present context, an aminothiol compound is intended to indicate a compound comprising at least one mercapto group (—S—H) and at least on amino group (—N—H).

In the present context, a dithiol compound is intended to indicate a compound comprising a dithiol

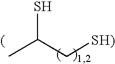

moiety.

### DESCRIPTION OF THE INVENTION

In one embodiment, the reaction is run at pH between 5.1 and 7.0, such as between 5.5 and 6.5, such as between 5.8 and 6.2, such as around 6.0.

In one embodiment, the dipolar solvent is N-methylpyrrolidinone (NMP), N,N-dimethylformamide, DMSO, 1,3-dimethylimidazolidin-2-one, 1,3-dimethyltetrahydropyrimidin-2-one, acetonitrile, propionitrile, N-methylformamide, formamide, N,N-dimethylacetamide or N-methylacetamide. In one embodiment, the dipolar solvent is selected from NMP, N,N-dimethylformamide and DMSO. Typically, the dipolar solvent is present in an amount from 10-95 (v/v) %, such as 10-70 (v/v) %, such as 10-50 (v/v) %, such as 10-30(v/v) %, such as 10-20 (v/v) %, such as 10-15 (v/v) %. Water typically makes up the remaining volume.

The conditions for the reaction of the invention may be achieved in several ways. In one embodiment, the GH derived aldehyde or ketone is dissolved in an optionally aqueous dipolar solvent and the pH is adjusted to between 1 and 7 followed by the addition of suitable alkoxylamine, hydrazine, 2-aminothiol or dithiol compound dissolved in water, in mixtures of water and a dipolar solvent, or in a dipolar solvent alone. Alternatively, the alkoxylamine, hydrazine, 2-aminothiol or dithiol compound may be dissolved in a suitable buffer, optionally comprising a dipolar solvent, so that a pH between 1 and 7 will result directly upon adding the solution of the alkoxylamine, hydrazine, 2-aminothiol or dithiol compound to the solution of the GH derived aldehyde or ketone in an optionally aqueous dipolar solvent. Alternatively, instead of a dipolar solvent a solution of a compound in water or other solvents may be used, which enhances the solubility of proteins. Such a compound may be urea, guanidine hydrochloride, methylguanidine hydrochloride, thiocyanate salts, perchlorate salts, sodium dodecyl sulfate, and the like.

Useful buffer systems include trifluoroacetic acid (TFA) together with an amine, such as e.g. 2-methyl pyridine. Another useful buffer system is acetic acid together with an amine, such as e.g. 2-methyl pyridine.

In one embodiment, GH is human growth hormone (hGH).

In one embodiment, GH is a variant of hGH, wherein a variant is understood to be the compound obtained by substituting one or more amino acid residues in the hGH sequence with another natural or unnatural amino acid; and/or by adding one or more natural or unnatural amino acids to the hGH sequence; and/or by deleting one or more amino acid residue from the hGH sequence, wherein any of these steps may optionally be followed by further derivatization of one or more amino acid residue. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine, lysine, histidine), acidic amino acids (such as glutamic acid and aspartic acid), polar amino acids (such as glutamine, cysteine and asparagine), hydrophobic amino acids (such as leucine, isoleucine, proline, methionine and valine), aromatic amino acids (such as phenylalanine, tryptophan, tyrosine) and small amino acids (such as glycine, alanine, serine and threonine.).

In one embodiment, GH has at least 80%, such as at least 85%, such as at least 90%, such as at least 95% identity with hGH. In one embodiment, said identities to hGH is coupled to at least 20%, such as at least 40%, such as at least 60%, such as at least 80% of the growth hormone activity of hGH as determined in assay I herein.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix:

BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for protein comparisons (along with no penalty for end gaps) using the GAP algorithm.

In one embodiment, GH is hGH extended with up to 100 amino acid residues at the N-terminal. In particular, said extension is up to 50, such as up to 40, such as up to 20, such as up to 10, such as up to 5, such as 1, 2, 4 or 5 amino acid residues.

In one embodiment, pl of the GH is similar to that of hGH, such as between 4.0 and 8.0, such as between 4.0 and 6.0, such as between 4.5 and 5.5. pl for GH may be calculated as described in M. Caplice, J. J. A. Heffron, Biochemical Education, 1988,16, 91-92.

Growth hormone derived aldehydes or ketones may be prepared by several routes.

One possibility for preparing GH-derived aldehydes is the periodate-mediated oxidation of an GH which contains an N-terminal serine or threonine. Such GH may be prepared by standard genetic modification of E. coli or other suitable cells to produce the desired, recombinant variant of GH. The serine or threonine added to GH, such as e.g. hGH need not be attached directly to the N-terminal. One or more amino acid residues may be inserted between the serine or threonine and the original N-terminal. In this embodiment, the resulting serine or threonine comprising GH may be described by the formula Z-XX-GH, such as Z-XX-hGH, wherein Z represent serine or threonine and XX represent any sequence of 0-50 amino acids. In particular, Z represents serine. In particular XX represents a sequence with up to 40, such as up to 20, such as up to 10, such as up to 5, such as 0, 1, 2, 3, 4 or 5 amino acid residues. Particular mentioning is made of Ser-hGH.

Alternatively, a GH-derived aldehyde, such as e.g. a hGH-derived aldehyde, may be prepared by periodate-mediated oxidation of a derivative of hGH, in which one or several of the available aspartic or glutamic acid residues has been used to acylate an amine of the general formula H₂N—R⁴—CH(XH)—CHR⁵13 XH, wherein R⁴ represents an organic diradical, R⁵ represents an organic radical, and each X independently represents O or NH. Such an acylation may be accomplished selectively by treating GH, such as e.g. hGH with an excess of said amine and a suitable enzyme, such as a glutamyl or aspartyl transpeptidase.

Alternatively, a GH-derived aldehyde or ketone, such as e.g. a hGH-derived aldehyde or ketone may be prepared by coupling a thiol of the general formula HS—R⁶—C(═O)—R⁷, wherein R⁶ represents an organic diradical, and R⁷ represents hydrogen or an organic radical, to one of the available tyrosine residues by means of a tyrosinase, e.g. a mushroom tyrosinase, as described in the literature (S. Ito et al., J. Med. Chem. 1981, 24, 673-677).

Alternatively, a GH-derived aldehyde or ketone, such as e.g. a hGH-derived aldehyde or ketone may be prepared by coupling a thiol of the general formula HS—R⁸—CH(XH)—CHR⁹—XH, wherein R⁸ represents an organic diradical, R⁹ represents hydrogen or an organic radical, and each X independently represents O or NH, by means of a tyrosinase, e.g. a mushroom tyrosinase, as described in the literature (S. Ito et al., J. Med. Chem. 1981, 24, 673-677), followed by periodate-mediated oxidation of the resulting product.

Alternatively, a GH-derived aldehyde or ketone, such as e.g. a hGH-derived aldehyde or ketone may be prepared by amide formation of the carboxy-terminal of GH, such as e.g. hGH with an unnatural α-amino acid amide, which contains a ketone or an aldehyde as side-chain functional group. Such an unnatural α-amino acid amide may be coupled with GH, such as e.g. hGH with the aid of an enzyme, such as a carboxypeptidase.

Alternatively, a GH-derived aldehyde or ketone, such as e.g. a hGH derived aldehyde or ketone, may be obtained by reductive alkylation of the N-terminal amino group with a compound comprising a moiety which subsequently can be transformed into an aldehyde or a ketone, see US 20040127417.

The alkoxyamine, hydrazine, aminothiol, or dithiol compounds, i.e. the compounds comprising the moiety ultimately conjugated to GH, used in the methods of the present invention improve one or more pharmacological property of the resulting conjugated GH compared to the un-conjugated GH. Particular mentioning is made of conjugating compounds comprising protein radicals, straight or branched PEG or mPEG radicals and amino derivatives thereof; straight, branched and/or cyclic C₁₋₂₂alkyl, C₂₋₂₂alkenyl, C₂₋₂₂alkynyl, C₁₋₂₂heteroalkyl, C₂₋₂₂heteroalkenyl, C₂₋₂₂heteroalkynyl, wherein one or more homocyclic aromatic compound biradical or heterocyclic compound biradical may be inserted, and wherein said C₁-C₂₂ or C₂-C₂₂ radicals may optionally be substituted with one or more substituents selected from hydroxyl, halogen, carboxyl, heteroaryl and aryl, wherein said aryl or heteroaryl may optionally be further substiututed by one or more substituents selected from hydroxyl, halogen, and carboxyl; steroid radicals; lipid radicals; polysaccharide radicals, e.g. dextrans; polyamide radicals e.g. polyamino acid radicals; PVP radicals; PVA radicals; poly(1-3-dioxalane); poly(1,3,6-trioxane); ethylene/maleic anhydride polymer; Cibacron dye stuffs, such as Cibacron Blue 3GA, and polyamide chains of specified length, as disclosed in WO 00/12587, which is incorporated herein by reference. The alkoxyamine, hydrazine, aminothiol or dithiol compounds may also comprise a combination of the above mentioned radicals, including more than one of the above mentioned radicals.

In one embodiment (embodiment i), said alkoxyamine, hydrazine, aminothiol or dithiol compound (i.e. conjugating compound) is a compound of formula I

R¹—W [I]

wherein W represents

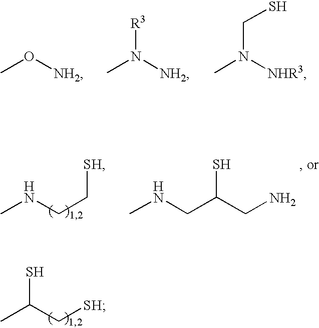

### R³ represents hydrogen or C₁₋₆ alkyl, such as methyl, ethyl, propyl, butyl, pentyl or hexyl;

### R¹ represents R²—R⁴—, wherein

### R² represents

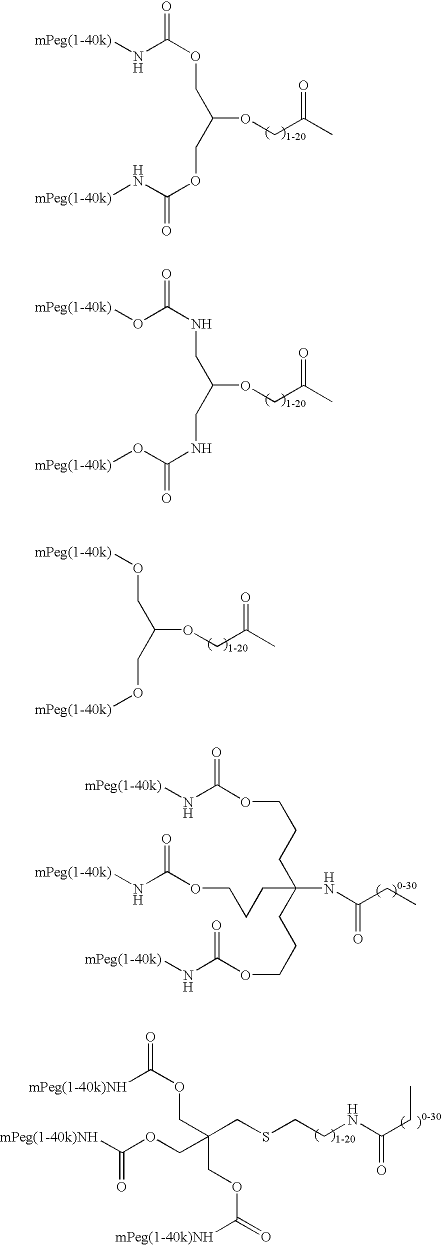

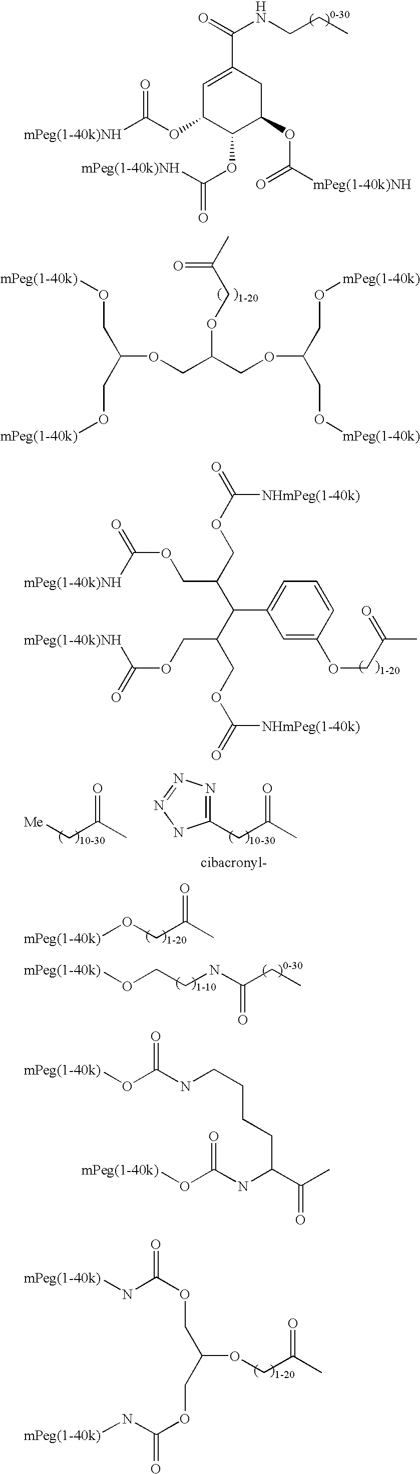

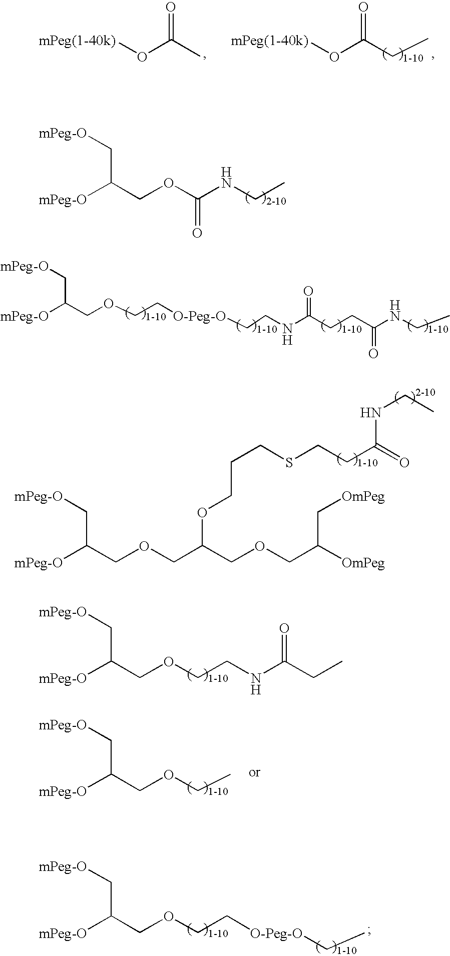

and R⁴ represents a bond or

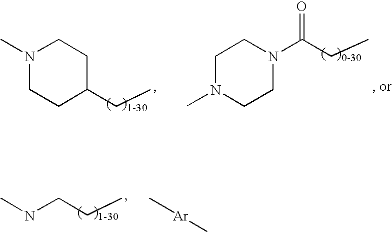

Ar=arylene, wherein Ar represents arylene or heteroarylene, both of which may optionally be substituted with one or more substituents selected from carboxy, hydroxyl, nitro or, cyano.

In embodiment (embodiment ii) according to embodiment i), W represents

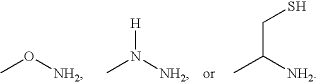

In one embodiment (embodiment iii) according to any or all of embodiment i) or ii), R² represents

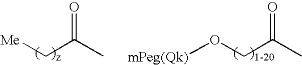

wherein Z is 14, 16, 18or 20,

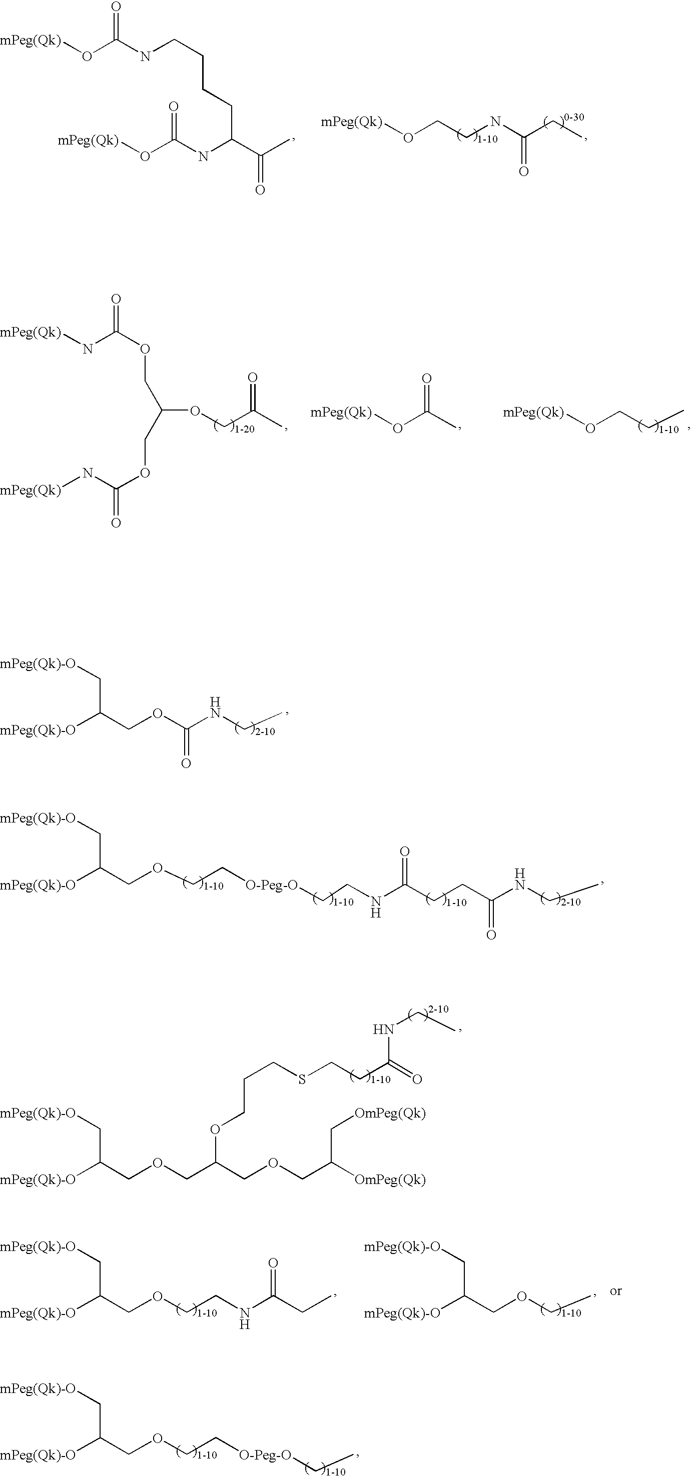

wherein Q represents an integer from 10-20, 10-30, 10-40, 20-30, 20-40, 30-40, such as 10, 20 or 30.

In one embodiment (embodiment iv) according to any or all of embodiment i), ii) or iii), R⁴ represents

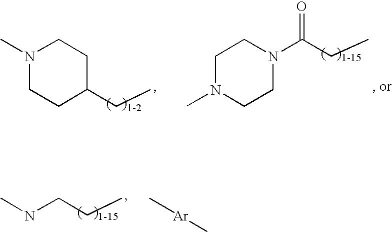

Ar=arylene, wherein Ar may represent pyridine biradical or nitro substituted phenylene.

In one embodiment (embodiment v) according to any or all of embodiment i), ii), iii), or iv), R² represents

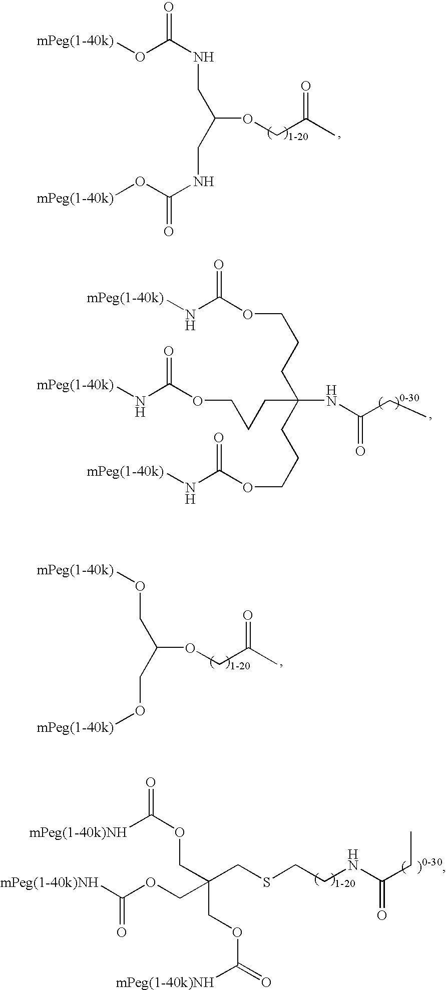

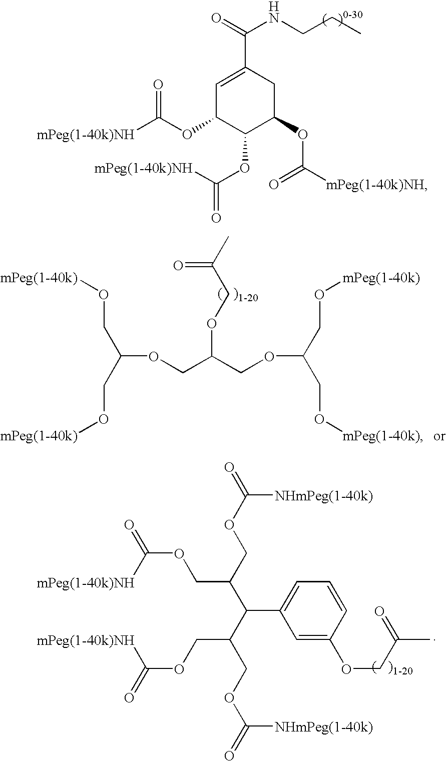

In one embodiment, the compound according to formula [I] is selected amongst

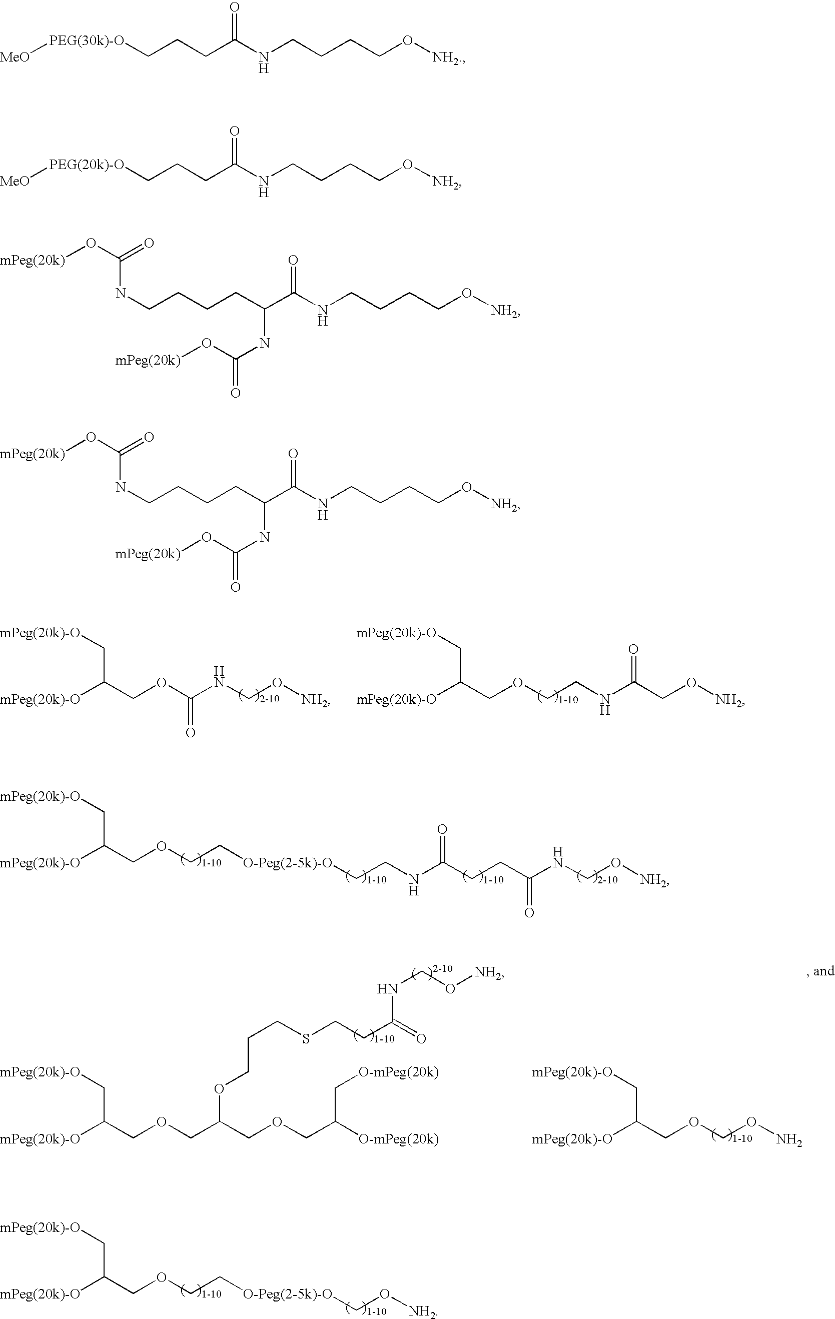

In one embodiment, the compound according to formula [I] is selected amongst

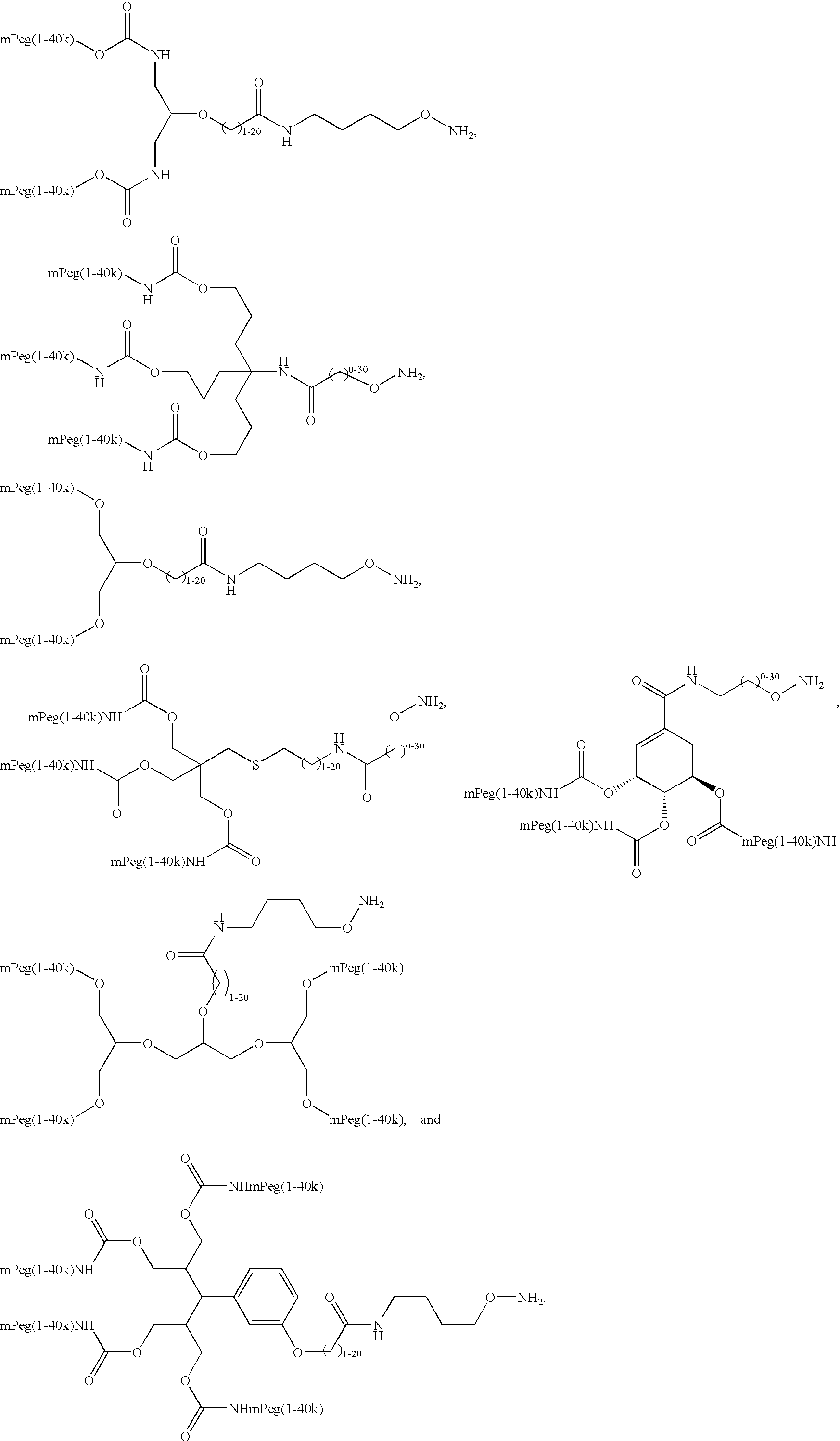

In one embodiment, the compound of formula [I] is selected amongst

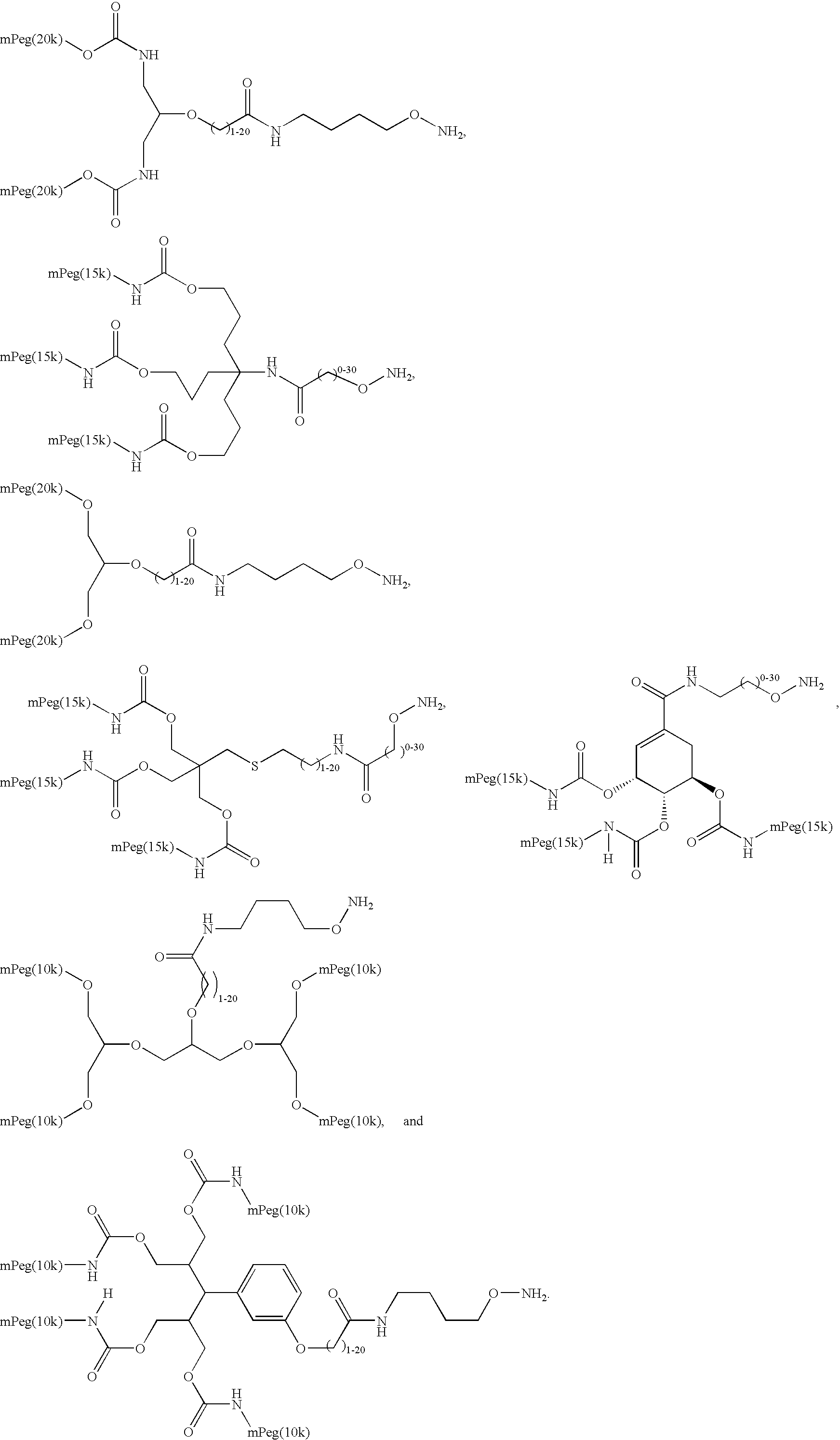

In one embodiment, the compound of formula [I] is selected amongst

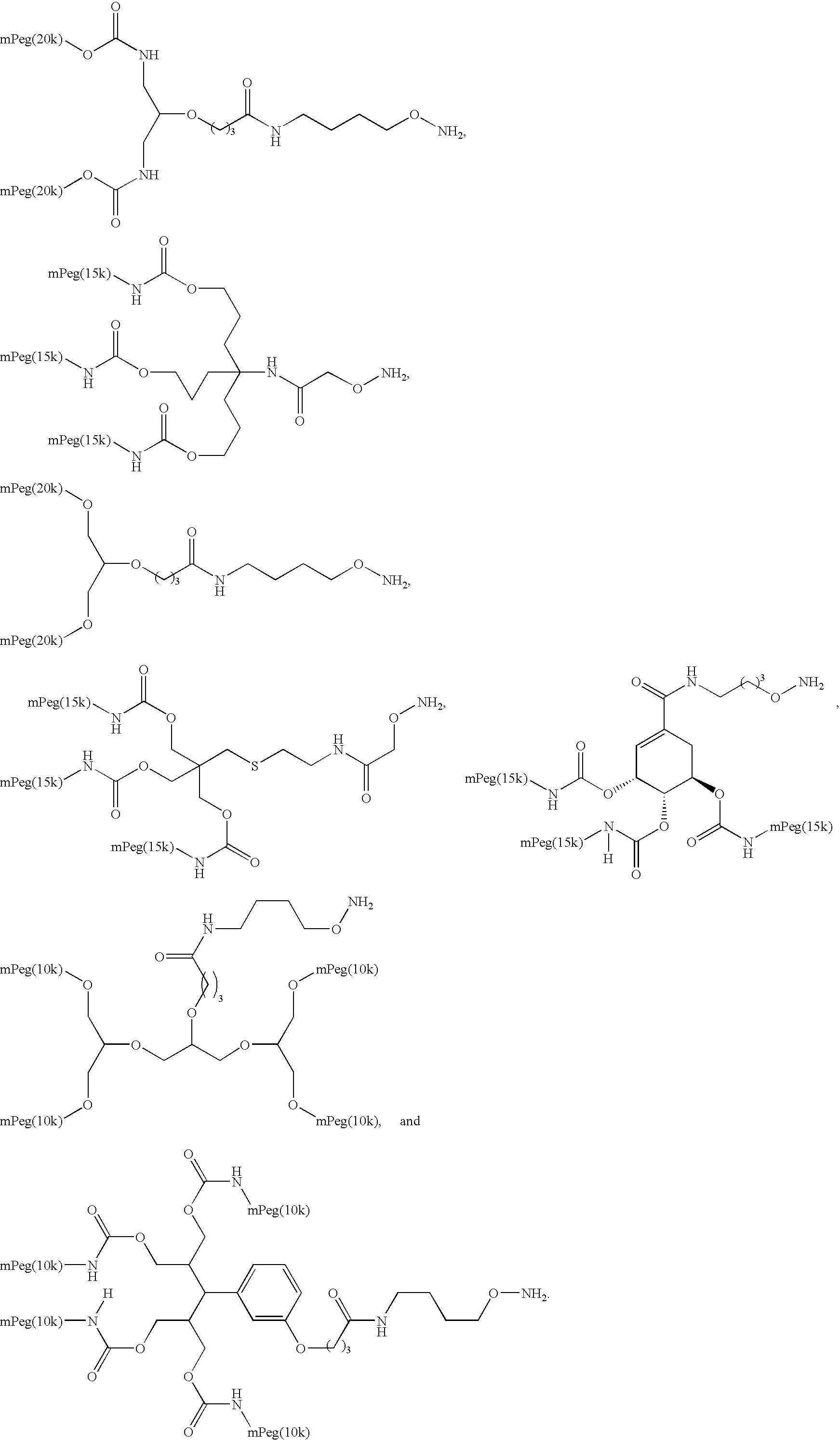

As described above, the present invention also provides conjugated GH compounds prepared by conjugating a compound of formula [I] to a GH (such as, e.g., hGH). Methods of conjugating PEG-molecules of formula [I] or similar to GH, GH derivatives, or other polypeptides are known in the art and described in, e.g., WO2005070468 (hereby incorporated by reference in its entirety), describing transglutaminase-mediated conjugation; and Gaertner et al., Bioconjugate Chem 1996;7:38-44, describing a method for N-terminal conjugation of proteins by generating an N-terminal aldehyde.

Accordingly, in one embodiment, the present invention provides a conjugated hGH compound selected amongst

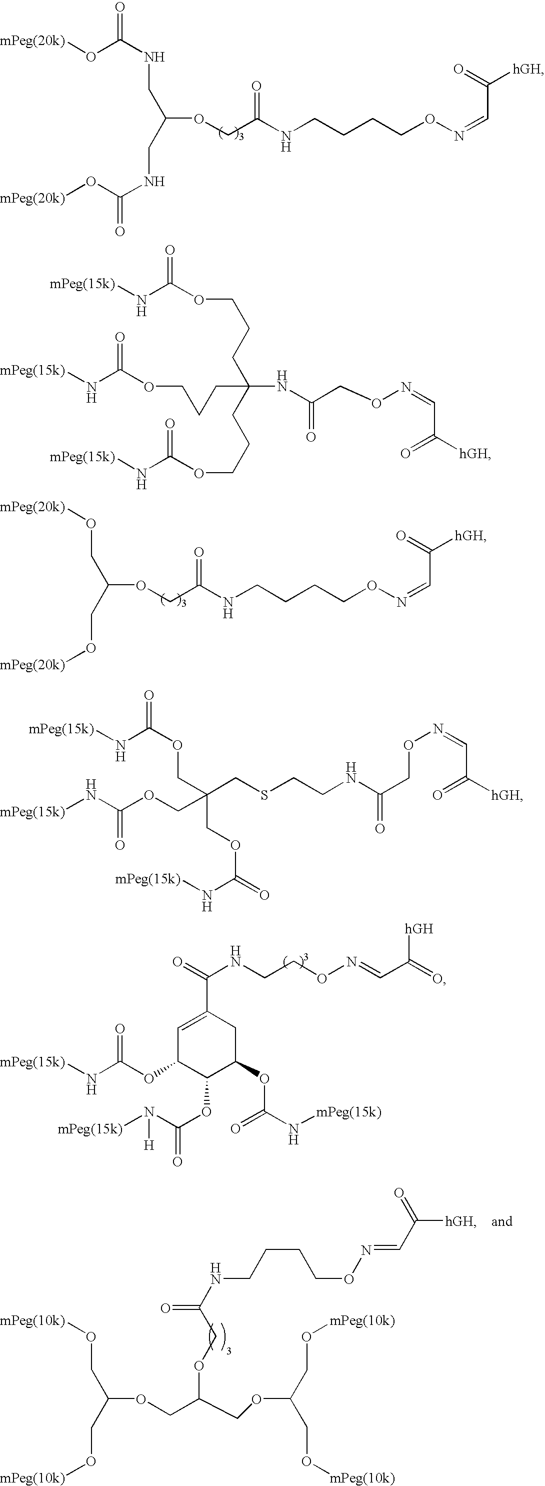

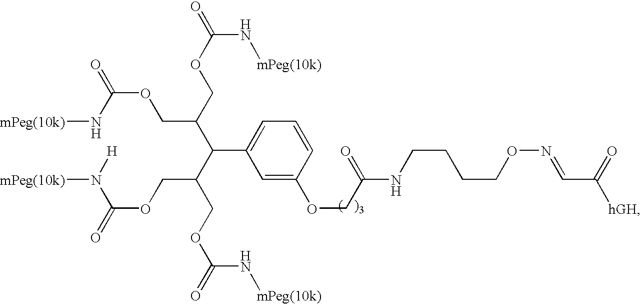

wherein GH represents the growth hormone radical obtained by removing —C(═O)—NH₂ from the side chain of a glutamine.

In one embodiment, the present invention provides a conjugated hGH compound selected amongst

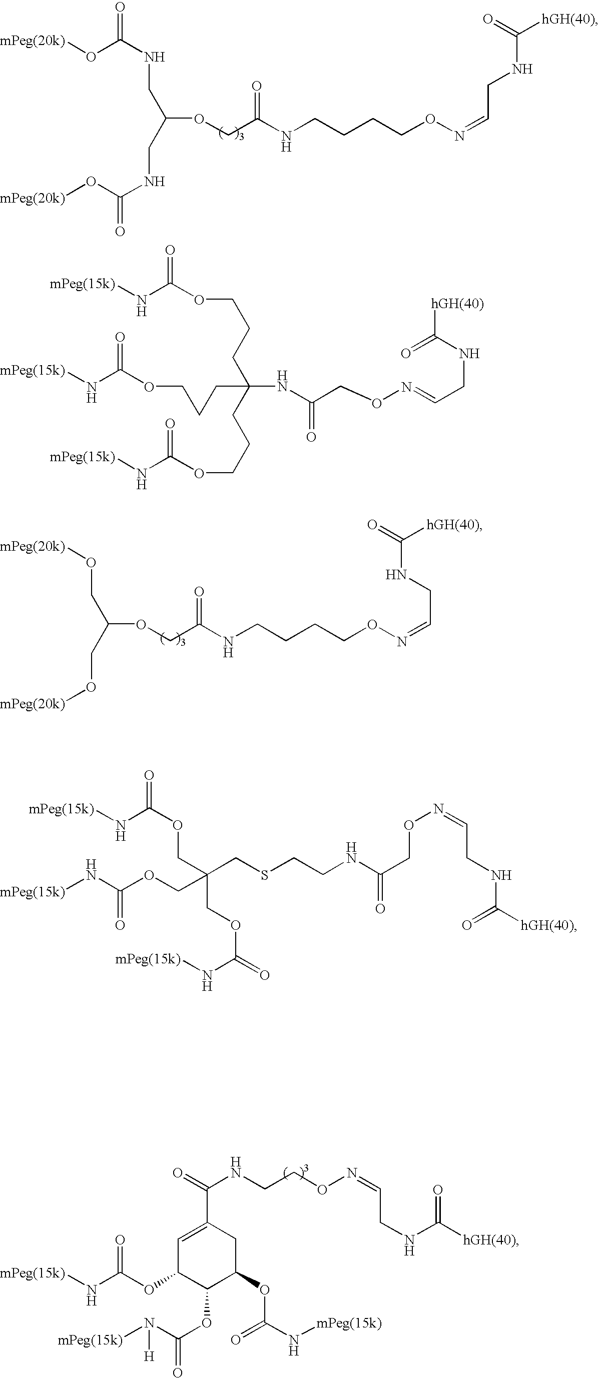

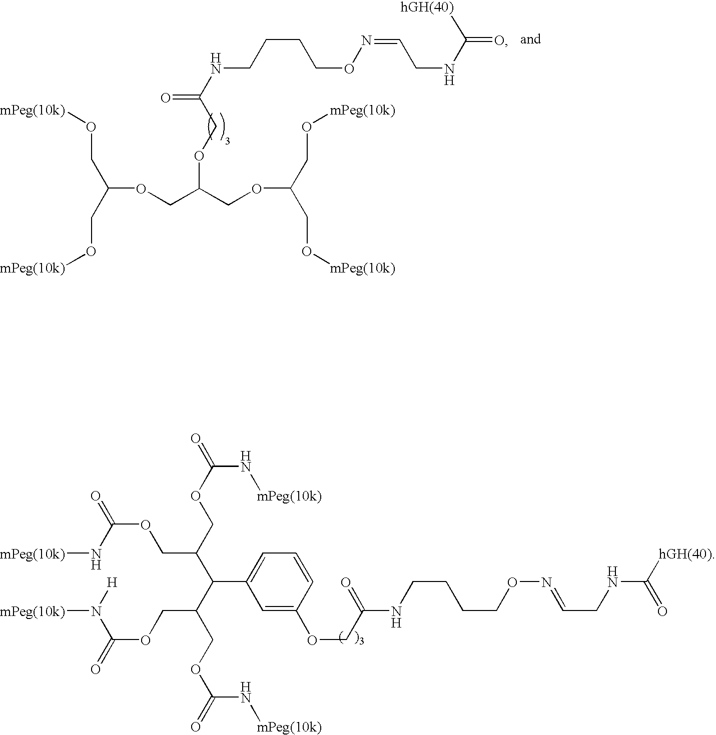

In one embodiment, the present invention provides a conjugated hGH compound selected amongst

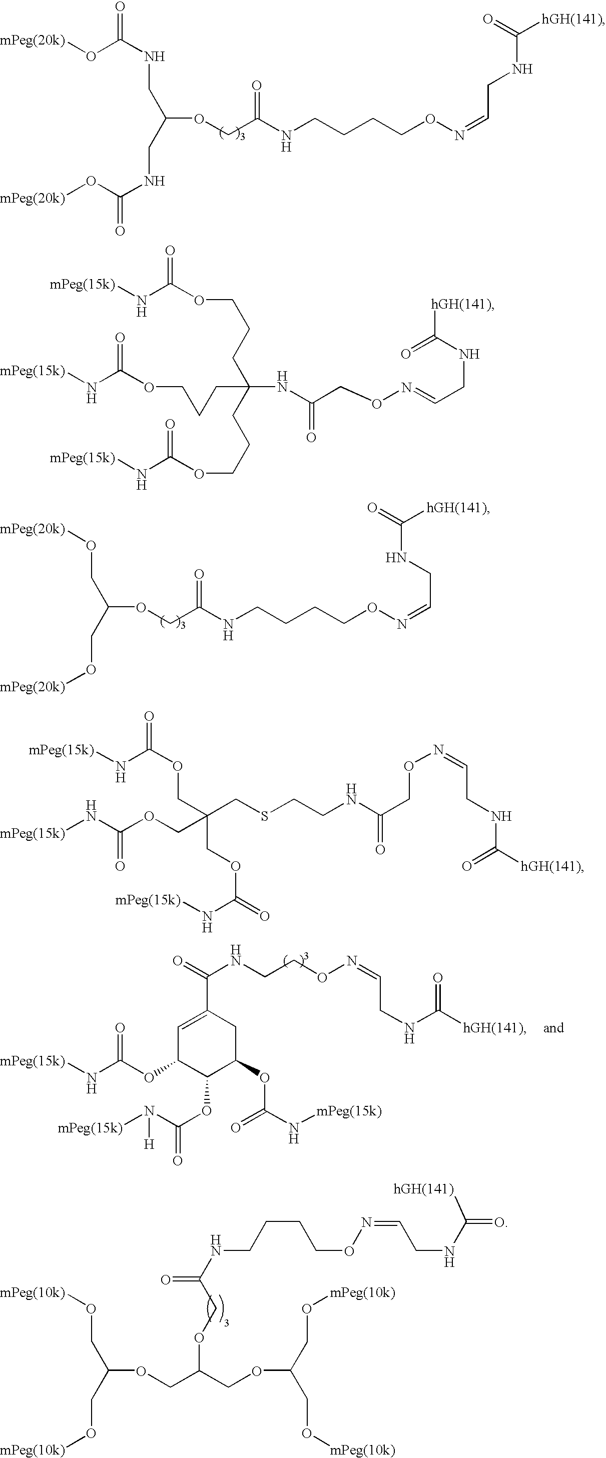

The alkoxyamino, hydrazine, aminothiol and dithiol compounds used in the methods of the present invention may be prepared as shown below.

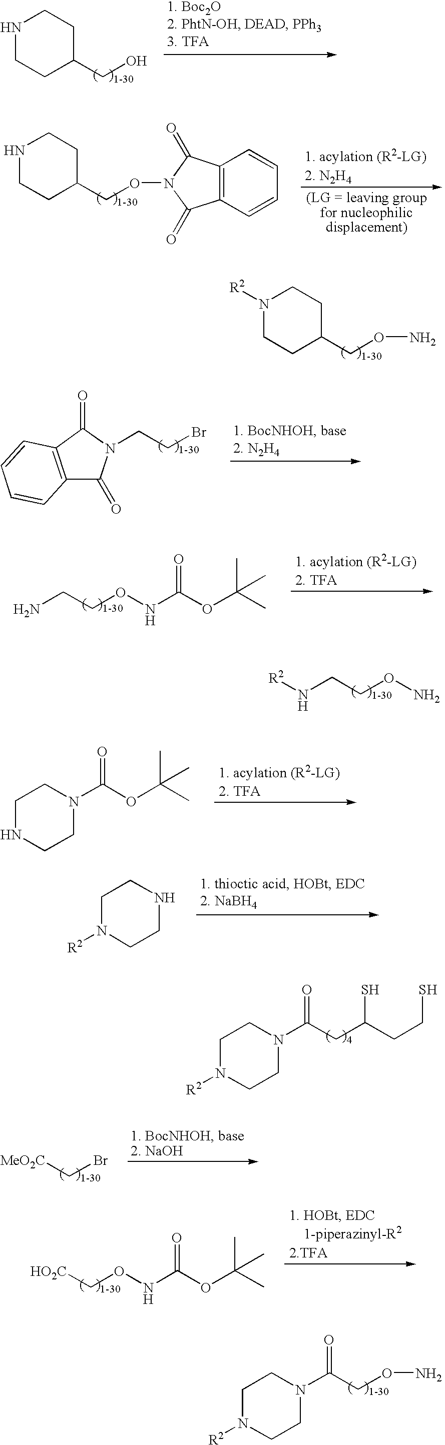

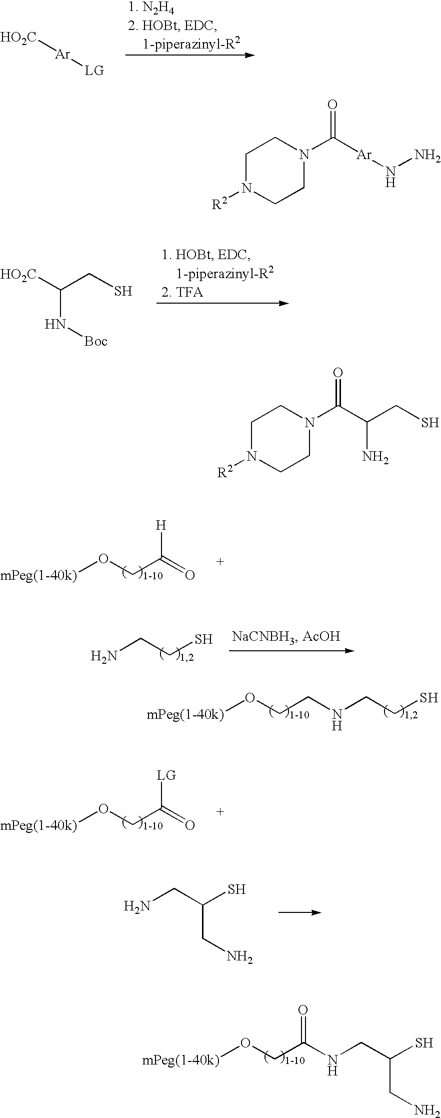

“LG” abbreviates leaving group.

As discussed above, the conjugation of GH results in improved pharmacological properties for the conjugated GH compared to the un-conjugated compound. Examples of such pharmacological properties include functional in vivo half-life, immunogencity, renal filtration, protease protection and albumin binding.

The term “functional in vivo half-life” is used in its normal meaning, i.e., the time at which 50% of the biological activity of the GH or conjugated GH are still present in the body/target organ, or the time at which the activity of the GH or GH conjugate is 50% of its initial value. As an alternative to determining functional in vivo half-life, “in vivo plasma half-life” may be determined, i.e., the time at which 50% of the GH or GH conjugate circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life.

The term “increased” as used in connection with the functional in vivo half-life or plasma half-life is used to indicate that the relevant half-life of the GH conjugate is statistically significantly increased relative to that of the parent GH, as determined under comparable conditions. For instance the relevant half-life may be increased by at least about 25%, such as by at lest about 50%, e.g., by at least about 100%, 150%, 200%, 250%, or 500%. In one embodiment, the compounds of the present invention exhibit an increase in half-life of at least about 5 h, such as at least about 24 h, such as at least about 72 h, such as at least about 7 days, relative to the half-life of the parent GH.

Measurement of in vivo plasma half-life can be carried out in a number of ways as described in the literature. An increase in in vivo plasma half-life may be quantified as a decrease in clearance (CL) or as an increase in mean residence time (MRT). Conjugated GH for which the CL is decreased to less than 70%, such as less than 50%, such than less than 20%, such than less than 10% of the CL of the parent GH as determined in a suitable assay is said to have an increased in vivo plasma half-life. Conjugated GH for which MRT is increased to more than 130%, such as more than 150%, such as more than 200%, such as more than 500% of the MRT of the parent GH in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable text animals include normal, Sprague-Dawley male rats, mice and cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques. Typically, the GH levels are measured indirectly by measuring the IGF-1 (insulin-like growth factor 1) level as described in Assay II herein.

The term “Immunogenicity” of a compound refers to the ability of the compound, when administered to a human, to elicit a deleterious immune response, whether humoral, cellular, or both. In any human sub-population, there may exist individuals who exhibit sensitivity to particular administered proteins. Immunogenicity may be measured by quantifying the presence of growth hormone antibodies and/or growth hormone responsive T-cells in a sensitive individual, using conventional methods known in the art. In one embodiment, the conjugated GH exhibits a decrease in immunogenicity in a sensitive individual of at least about 10%, preferably at least about 25%, more preferably at least about 40% and most preferably at least about 50%, relative to the immunogenicity for that individual of the parent GH.

The term “protease protection” or “protease protected” as used herein is intended to indicate that the conjugated GH is more resistant to the plasma peptidase or proteases than is the parent GH. Protease and peptidase enzymes present in plasma are known to be involved in the degradation of circulating proteins, such as e.g. circulating peptide hormones, such as growth hormone.

Resistance of a protein to degradation by for instance dipeptidyl aminopeptidase IV (DPPIV) is determined by the following degradation assay: Aliquots of the protein (5 nmol) are incubated at 37+ C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the protein degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999;79:93-102 and Mentlein et al. Eur. J. Biochem. 1993;214:829-35. Proteins and their degradation products may be monitored by their absorbance at 220 nm (peptide bonds) or 280 nm (aromatic amino acids), and are quantified by integration of their peak areas related to those of standards. The rate of hydrolysis of a protein by dipeptidyl aminopeptidase IV is estimated at incubation times which result in less than 10% of the peptide being hydrolysed. In one embodiment, the rate of hydrolysis of the GH conjugate is less than 70%, such as less than 40%, such as less than 10% of that of the parent GH.

The most abundant protein component in circulating blood of mammalian species is serum albumin, which is normally present at a concentration of approximately 3 to 4.5 grams per 100 milliters of whole blood. Serum albumin is a blood protein of approximately 70,000 daltons which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. Serum albumin has a half-life of more than one week, and one approach to increasing the plasma half-life of proteins has been to conjugate to the protein a group that binds to serum albumin. Albumin binding property may be determined as described in J.Med.Chem, 43, 2000, 1986-1992, which is incorporated herein by reference.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the claims appended hereto as permitted by applicable law.

### EXAMPLES

In the examples the following terms are intended to have the following, general meanings:
Boc: tert-butyloxycarbonyl
DCM: dichloromethane, methylenechloride
DMF: N,N-dimethyl formamide
DMSO: dimethyl sulfoxide
DMPU: 1,3-dimethyltetrahydropyrimidin-2-one
EDC: N-ethyl-N′-(3-dimethylaminopropyl)carbodiimide hydrochloride
HOBt: N-hydroxybenzotriazole, 1-hydroxybenzotriazole
NMP: N-methylpyrrolidone
HPLC: high pressure liquid chromatography
r.t. room temperature
TFA trifluoroacetic acid

### Example 1

### (mPEG-30k)-hGH

### Step 1: mPEG-30k-alkoxylamine

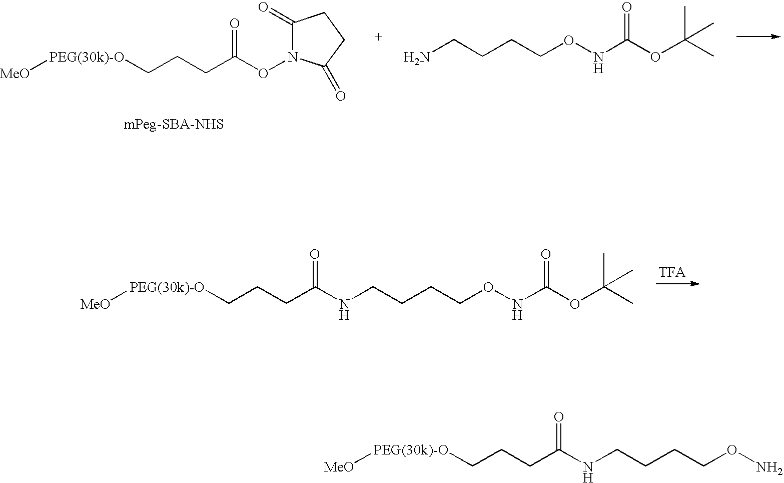

To a solution of 4-(N-Boc-aminoxy)butylamine (0.43 g, 2.10 mmol) in DCM (40 ml) was added mPEG(30K)-SBA-NHS (5.0 g, 0.17 mmol). The resulting mixture was stirred at room temperature for 5 d, concentrated under reduced pressure, and the residue was dried in vacuum. Recrystallization from iPrOH (4×80 ml) followed by coevaporation with DCM and drying under reduced pressure yielded 4.14 g of the Boc-protected alkoxylamine.

To this product (0.42 g, approx 14 μmol, 16 eq) were added DCM (15 ml) and TFA (15 ml). After 0.5 h the mixture was concentrated, the residue redissolved in DCM (5 ml) and toluene (80 ml) and concentrated again, and dried under reduced pressure overnight. The residue was redissolved in DCM, distributed equally into two vials, concentrated and dried in vacuum. Water (2.1 ml) was added to each of the vials, and with 2-methylpyridine (50 μl) the pH was adjusted to 6.

### Step 2: Ser-hGH

The Ser-hGH analogue expression plasmid was created on the basis of pNNC13 (Zbasic2mt-D4K-hGH), which expresses the wild type hGH in fusion with Zbasic domain (mvdnkfnkerrrarreirhlpnlnreqrrapirslrddpsqsanllaeakklnraqapkyrggsddddksfptiplsrlfdnamlrah rlhqlafdtyqefeeayipkeqkysflqnpqtslcfsesiptpsnreetqqksnlellrisllliqswlepvqflrsvfanslvygasdsnvy dllkdleegiqtlmgrledgsprtgqifkqtyskfdtnshnddallknygllycfrkdmdkvetfrivqcrsvegscgf (SEQ ID NO:2)). Additional Ser was inserted in front of Phe, the first amino acid of mature hGH, by QuikChange® XL Site-Directed Mutagenesis Kit from Stratagene with a pair of primes: 5′ end: pNNC13 Ser-F

(SEQ ID NO:3)

5′-GGATCAGACGACGACGACAAAagcTTCCCAACCATTCCCTTATCC-
3′
and

(SEQ ID NO:4)

3′end: pNNC13 Ser-R
5′-GGATAAGGGAATGGTTGGGAAgctTTTGTCGTCGTCGTCTGATCC-
3′.

E. coli BL21 (DE3) was transformed by pET11a-Zbasic2mt-D4K-Ser-hGH. Single colony was inoculated into 100 ml LB media with 100 μg/ml Amp and grown at 37° C. When OD600 reached 0.6, the cell culture temperature was reduced to 30° C., and the cells were induced with 1 mM IPTG for 4 hours at 30 degree. The bacteria cells were harvested by centrifugation at 3000 g for 15 minutes (Eppendorf centrifuge 5810R). The cell pellet was re-suspended in cell lysis buffer (25 mM Na₂HPO₄ 25 mM NaH₂PO₄ pH 7, 5 mM EDTA, 0.1% Triton X-100), and the cells were disrupted by cell disruption at 30 kpsi (Constant Cell Disruption Systems). The lysate was clarified by centrifugation at 10,000 g for 30 minutes. The supernatant was saved and used for purification, while the pellet was discarded.

Zbasic2mt-D4K-Ser-hGH was purified on SP-Sepharose using a step gradient elution (buffer A: 25 mM Na₂HPO₄ 25 mM NaH₂PO₄ pH 7; buffer B: 25 mM Na₂HPO₄ 25 mM NaH₂PO₄ pH 7, 1 M NaCl). The protein was subsequently cleaved using Enteropeptidase for the release of Ser-hGH. Ser-hGH was further purified on a Butyl Sepharose 4FF column to separate the product from the Zbasic2mt-D4K domain and Enteropeptidase (buffer A: 100 mM Hepes pH 7.5; buffer B: 100 mM Hepes pH 7.5, 2 M NaCl, a linear gradient was used). The final product of Ser-hGH was buffer exchanged and lyophilized from 50 mM NH₄HCO₃, pH 7.8.

### Step 3: Oxidation and Oximation of Ser-hGH

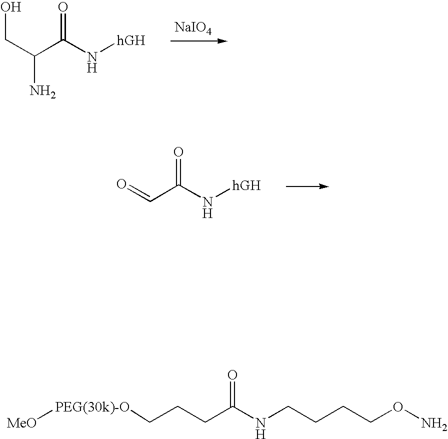

A buffer (pH 8±1) was prepared by dissolving triethanolamine (0.24 g, 1.61 mmol) and methionine (1.52 g, 10.2 mmol) in water (100 ml), and a solution of sodium periodate (5.3 mg, 24.7 μmol) in water (1.0 ml) was prepared.

Into two vials with SerhGH (10 mg, 450 nmol in each vial) were added the buffer (1.3 ml) and then the sodium periodate solution (0.15 ml, 7.8 eq). After 15 min cold DMF (0.6 ml at approx 0° C.) was added to each of the vials, and the resulting, clear solutions were added to the PEG-solutions prepared in Step 1. Almost clear solutions (pH=6) result. The mixtures were kept at room temperature for 94 h. Then to each of the vials the buffer (4 ml) was added, the mixtures were pooled, and the product was purified by ion-exchange chromatography using a MonoQ High Resolution 10/10 column. This purification gave 11 mg (23% yield) of (mPeg-30k)-hGH.

### PHARMACOLOGICAL METHODS

### Assay (I) BAF-3GHR Assay to Determine Growth Hormone Activity

The BAF-3 cells (a murine pro-B lymphoid cell line derived from the bone marrow) was originally IL-3 dependent for growth and survival. II-3 activates JAK-2 and STAT which are the same mediators GH is activating upon stimulation. After transfection of the human growth hormone receptor the cell line was turn into a growth hormone-dependent cell line. This clone can be used to evaluate the effect of different growth hormone samples on the survival of the BAF-3GHR.

The BAF-3GHR cells are grown in starvation medium (culture medium without growth hormoen) for 24 hours at 37° C., 5% CO₂.

The cells are washed and re-suspended in starvation medium and seeded in plates. 10 μl of growth hormone compound or human growth hormone in different concentrations or control is added to the cells, and the plates are incubated for 68 hours at 37° C., 5% CO₂.

AlamarBlue® is added to each well and the cells are then incubated for another 4 hours. The AlamarBlue® is a redox indicator, and is reduced by reactions innate to cellular metabolism and, therefore, provides an indirect measure of viable cell number.

Finally, the metabolic activity of the cells is measure in a fluorescence plate reader. The absorbance in the samples is expressed in % of cells not stimulated with growth hormone compound or control and from the concentration-response curves the activity (amount of a compound that stimulates the cells with 50%) can be calculated.

### Assay (II) IGF-1 ELISA Assay

IGF-1 in rat or mouse plasma or serum is determined in a two-site immunoenzymometric assay in an OCTEIA™ kit obtainable from IDS Ltd., Boldon, England.

The samples are treated so as to inactivate the binding protein, IGF-BP 1-6. In the OCTEIA kit, a purified monoclonal anti-rat IGF-I is coated onto the inner surface of microtitre wells. The treated, diluted samples are incubated together with biotinylated polyclonal rabbit anti-rat IGF-I in the wells for two hours. The wells are then washed and horseradish peroxidase labelled avidin is added. After a further wash, a chromogenic compound, tetramethylbenzidine, is added to develop colour. The colour of the stopped reaction is read in a microtitre plate reader, where the colour intensity is directly proportional to the amount of rat or mouse IGF-I present in the ample.

A similar assay with minor modifications can be used to determine human IGF-I.

1. A method for the production of a conjugated growth hormone (GH) with improved pharmacological properties compared to the un-conjugated GH, the method comprising reacting a growth hormone derived aldehyde or ketone with a compound selected from the group consisting of: alkoxyamine, hydrazine, aminothiol and dithiol at pH 1-7 in the presence of a dipolar solvent.

2. The method according to claim 1, wherein the pH is between about 5.1 and about 7.0.

3. The method according to claim 1, wherein the pH is about 6.

4. The method according to claim 1, wherein the dipolar solvent is selected from the group consisting of: N-methylpyrrolidinone, N,N-dimethylformamide, DMSO, 1,3-dimethylimidazolidin-2-one, 1,3-dimethyltetrahydropyrimidin-2-one, acetonitrile, propionitrile, N-methylformamide, formamide, N,N-dimethylacetamide or N-methylacetamide.

5. The method according to claim 4, wherein the dipolar solvent is selected from the group consisting of: N-methylpyrrolidinone, NN-dimethylformamide or DMSO.

6. The method according to claim 1, wherein the dipolar solvent is present in an amount between 10 (v/v) % and 95 (v/v) %.

7. The method according to claim 1, wherein said growth hormone derived aldehyde or ketone is reacted with an alkoxyamine compound.

8. The method according to claim 1, wherein said growth hormone derived aldehyde or ketone is reacted with a hydrazine compound.

9. The method according to claim 1, wherein said growth hormone derived aldehyde or ketone is reacted with a 2-aminothiol compound.

10. The method according to claim 1, wherein said growth hormone derived aldehyde or ketone is reacted with a compound selected from the group consisting of: 1,2- and 1,3-dithiol.

11. The method according to claim 1, wherein said growth hormone derived aldehyde or ketone is a human growth hormone derived aldehyde or ketone.

12. The method according to claim 1, wherein said growth hormone comprises a conjugated growth hormone.

13. The method according to claim 20, wherein conjugated growth hormone is (mPEG-30k)-hGH.

14. The method according to claim 1, wherein said alkoxyamine, hydrazine, aminothiol or dithiol is a compound of the formula [I]
R¹—W [I]
wherein W represents 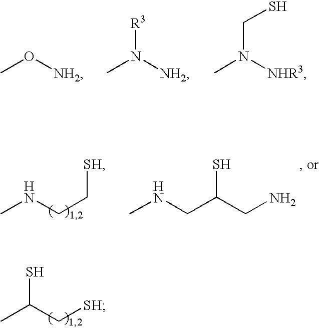
R³ represents hydrogen or C₁₋₆ alkyl;
R¹ represents R²-R⁴—, wherein
R² represents 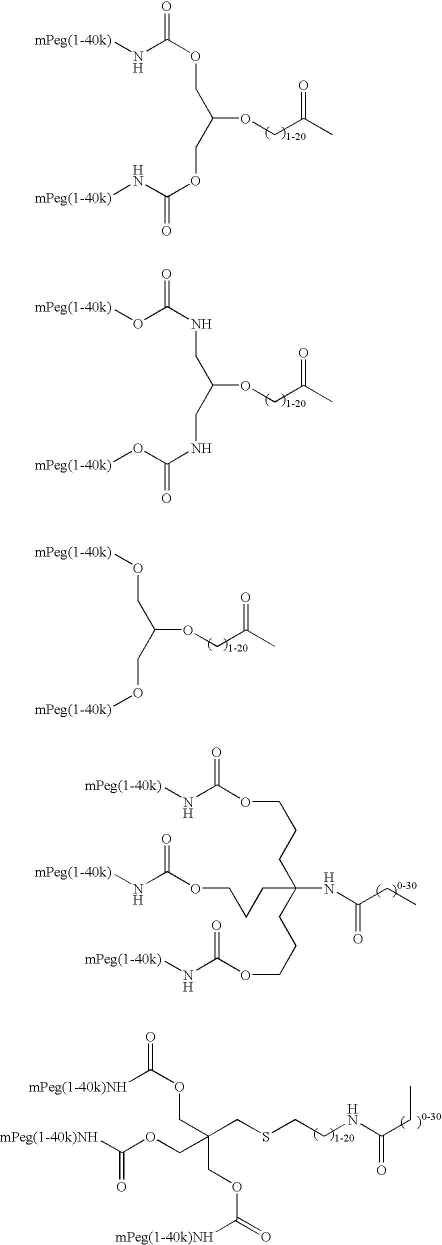 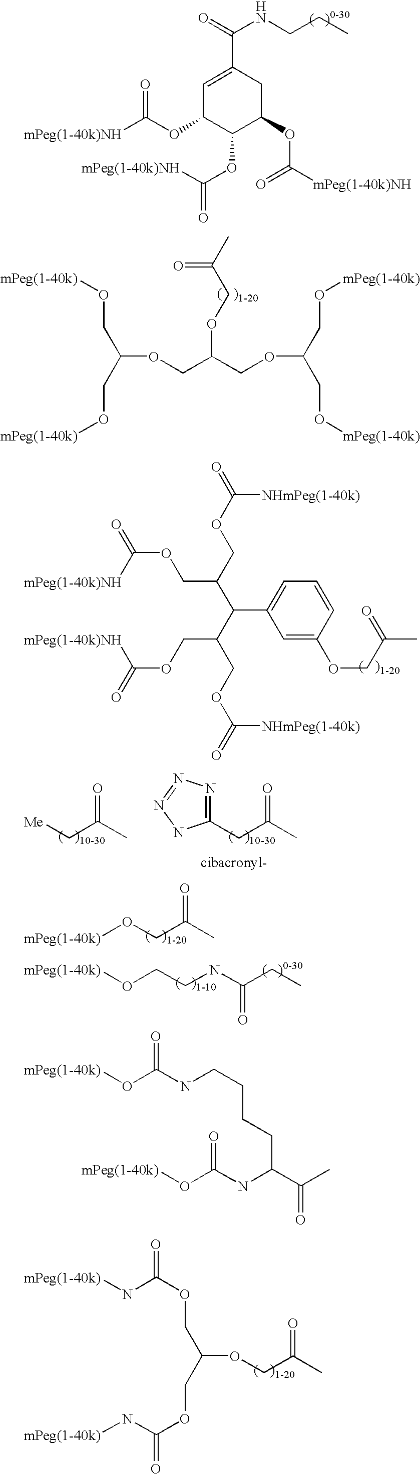 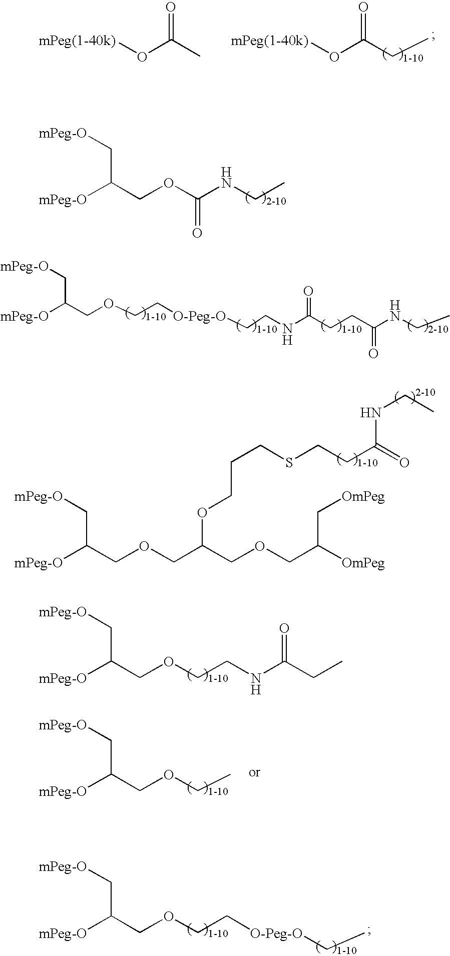
and R⁴ represents a bond or 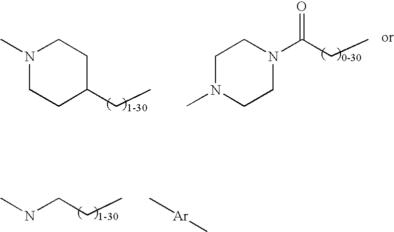
Ar=arylene, wherein Ar represents arylene or heteroarylene, both of which may optionally be substituted with one or more substituents selected from carboxy, hydroxy, nitro and cyano.

15. The method according to claim 14, wherein W represents

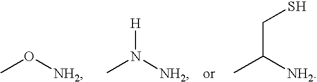

16. The method according to claim 14, wherein R² represents 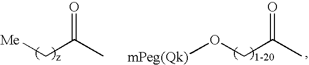
wherein Z is 14, 16, 18 or 20, 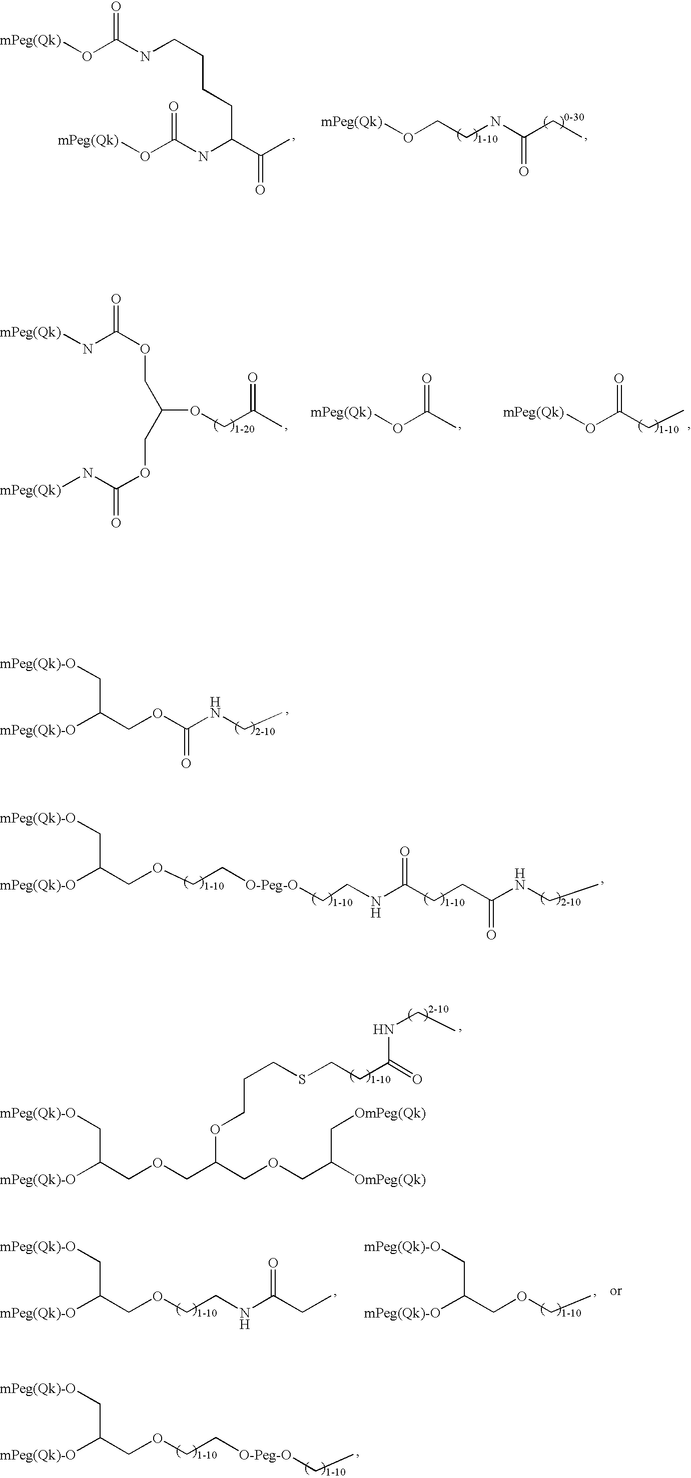
wherein Q represents an integer in a range selected from 10-20, 10-30, 10-40, 20-30, 20-40, and 30-40, such as 10, 20 or 30.

17. The method according to claim 14, wherein R⁴ represents 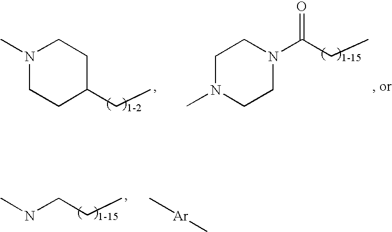
Ar=arylene, wherein Ar may represent pyridine biradical or nitro substituted phenylene.

18. The method according to claim 14, wherein the compound of formula [I] is selected from the group consisting of:

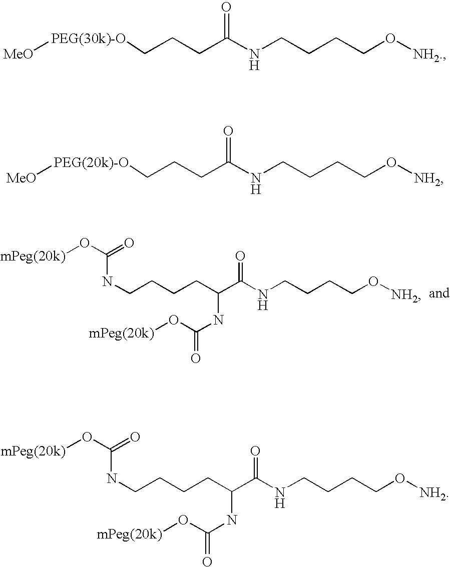

19. The method according to claim 14, wherein said compound of formula [I] is reacted with a hGH derived aldehyde or ketone.

20. The method according to claim 12, wherein said conjugated growth hormone is a branched or un-branched PEG or mPEG moiety.
